(19) United States
(12) Patent Application Publication
(10) Pub. No.: US 2018/0369615 A1
(43) Pub. Date: Dec. 27, 2018

(54) EXFOLIANT BAR

(71) Applicant: DPP PHARMACEUTICALS PTY LTD, Melbourne, Victora (AU)

(72) Inventor: ANGELO PARISI, Watsonia North, Victoria (AU)

(21) Appl. No.: 16/063,532

(22) PCT Filed: Dec. 16, 2016
(86) PCT No.: PCT/AU2016/051251

§ 371 Date: Jun. 18, 2018

(30) Foreign Application Priority Data
Dec. 18, 2015 (AU) 2015101768
Dec. 18, 2015 (AU) 2015258350

(51) Int. Cl. A61Q 19/10; A61K 8/60; A61K 8/02; A61K 8/92; A61K 8/9794

(52) U.S. Cl. CPC A61Q 19/10; A61K 8/60; A61K 8/29; A61K 8/922; A61K 8/9794; A61K 8/0216

(57) ABSTRACT

A single use body scrub bar comprising a substantially uniform mixture of exfoliant granules; and one or more binders.

### FIELD OF THE INVENTION

The present invention relates to exfoliant compositions. In particular, the present invention relates to an exfoliant composition in the form of an exfoliant bar.

### BACKGROUND OF THE INVENTION

The skin of the human body benefits from periodic cleansing with exfoliants in order to remove sebaceous secretions, soil deposits and dead skin. Exfoliation is the cosmetic practice of removing dead skin cells from the epidermis, the top layer of the skin. Human skin can shed 30,000 to 40,000 dead cells per minute naturally; however, many people need exfoliation products to help reduce acne and smooth skin.

Exfoliation can be accomplished in a variety of ways including (1) use of a physical abrasive such as a pumice stone, loofah, an exfoliating sponge, glove or towel, or a brush; or (2) by use of an exfoliant composition that contains sugar, salt or another natural or artificial exfoliating substance.

Common exfoliant compositions contain an abrasive component as the exfoliating substance. Simple exfoliant compositions provide the abrasive component as a loose material which can then be applied directly to the body. For example, Frank Body offers a loose coffee scrub comprising coffee seed powder, and Mr Bean Coffee Bean Scrub is a loose powder of coffee, sugar and salt. These compositions have some drawbacks with the key one being that, as they are a loose material, they can be messy to use as they can spill when applied and there is often a high degree of waste where the material is washed away without exfoliating the body. It can also be difficult to apply these loose materials to a specific area of the body, especially when standing in a shower.

Other exfoliant compositions are soap bars which comprise an abrasive component suspended in a soap component. As the abrasive components are dispersed throughout the soap bar, it can be difficult to achieve deep exfoliation of the skin. Some exfoliant compositions suspend the abrasive component in a gel or cream base to enable deeper exfoliation of the skin but provide some adhesiveness to address the physical difficulties of the scrub. However, there will still be a significant amount of exfoliant material which is lost before it can be used.

### SUMMARY OF THE INVENTION

According to a first aspect of the present invention, there is provided a single use body scrub bar comprising: exfoliant granules; and one or more holdings compounds, wherein, in use with water, the holding compounds will slowly release the granules over a predetermined period.

The exfoliant granules can be made of one or more materials. A person skilled in the art will know of the many options of materials which can be used as exfoliant granules. Examples include, but are not limited to, one or more of ground coffee beans, coffee seed powder, sugar crystals, salt crystals, ground oats, green tea, nut husks, sane, ground pumice, oatmeal, shredded coconut, micro beads and combinations thereof. A person skilled in the art will be aware that some exfoliant granules may be water soluble, such as sugar and salt.

A person skilled in the art will know of the many options which can be used as holding compounds. Examples include, but are not limited to, beeswax, cocoa butter, oat flour and shea butter.

The predetermined period is selected depending on the intended use of the body scrub bar. For example, a body scrub bar designed for a shower may release the granules faster than a body scrub bar designed for use in a bath. For example, the body scrub bar according to the invention will release the granules over approximately 10 minutes.

According to a second aspect of the present invention, there is provided a single use body scrub bar comprising a substantially uniform mixture of: exfoliant granules; and a binder.

A person skilled in the art will know of the many options which can be used as the binder. Examples include, but are not limited to, various grain derived flours or meals such as oat flour. Avena sativa leaf powder (also known as colloidal oatpowder) is an example of a suitable oat flour.

In embodiments comprising exfoliant granules made of two or more materials, the exfoliant granules are substantially the same size. Using granules of approximately the same size results in a bar that more readily maintains its compressed form.

In an embodiment of the present invention, the bar further comprises one or more emollients.

A person skilled in the art will know of the many options which can be used as the emollients. Examples include, but are not limited to, cocoa seed butter and various vegetable based oils such as olive oil, almond oil, coconut oil, and vitamin E oil.

In another embodiment of the present invention, the bar further comprises a lubricant. Examples include, but are not limited to, silicone ingredients such as polymethylsilsesquioxane.

In a further embodiment of the present invention, the bar further comprises a parfum. Examples include, but are not limited to, orange oil, natural coconut cream, vanilla, caramel fragrance, chocolate caramel fragrance, and berry fragrance.

Optionally, the bar may also comprise a colourant, for example, titanium dioxide.

In one embodiment, the bar is soap free.

In another aspect of the present invention there is provided a single use body scrub bar comprising a substantially uniform mixture (e.g. a mixture whereby the components are evenly dispersed) of: sucrose; exfoliant granules selected from the group of: coffee seed powder, salt, green tea or nut husks; a binder; one or more emoliants; and polyrnethylsilsesquioxane.

The description of the exfoliant bar as a “single use” bar is not limiting and could extend to any bar which it intended to be used for a single use.

Also disclosed herein is an exfoliant bar composition comprising exfoliant granules and binding particles. The binding particles may be capable of binding the exfoliant granules in the exfoliant bar composition together on pressing of the exfoliant bar composition.

In some forms, the composition comprises greater than 50% exfoliant granules by total weight of the composition.

In some forms, the exfoliant granules comprises a mixture of coffee grounds, sucrose particles and/or salt particles.

In some forms, an average particle size of the mixture of exfoliant granules is between 0.5 and 1 nm in diameter, and the average particle size of each of the exfoliant granules is substantially the same.

In some forms, the binding particles comprises avena sativa leaf powder particles. In some forms, the composition is in dry powder form.

In some forms, the binding particles comprises a wax. In some forms, the composition is in a wet form.

Also disclosed herein is a method of forming an exfoliant bar having a matrix containing exfoliant granules and binding particles. The method may comprise the steps of: providing in a mould a mixture containing the exfoliant bar composition as described above; and pressing the mixture in a mould to bind the mixture to form the exfoliant bar shaped by the mould.

In some forms, the mixture contains additional binding particles to that contained in the exfoliant bar composition. In some forms, the mixture contains emollients.

In some forms, the mixture is pressed at a pressure of between 50 and 100 psi for form the exfoliant bar. In other forms, the mixture is pressed at a pressure of 100 psi and above.

Also disclosed herein is a single use soap free body scrub bar. The single use soap free body scrub bar comprising a substantially uniform mixture of: exfoliant granules made of one or more materials selected from the group of: coffee seed powder, sugar, salt, green tea and nut husks; and one or more binders configured to bind the exfoliant granules together on pressing of the mixture; wherein, in use with water, the one or more binders will slowly release the granules over a predetermined period.

A detailed description of one or more embodiments of the invention is provided below. While the invention is described in connection with such embodiments, it should be understood that the invention is not limited to any embodiment. On the contrary, the invention encompasses numerous alternatives, modifications and equivalents.

### DETAILED DESCRIPTION

The present invention will now be described with reference to one or more embodiments of the invention.

The present invention resides in the ability to prepare a scrub bar in which the exfoliating granules bind together and keep their form, but are also released over a predetermined period to enable exfoliation of the user. Where emollients are used, they also need to bind together and assist the bar to retain its form.

An embodiment of the present invention provides a single use body scrub bar. The bar comprises exfoliant granules, such as coffee seed powder, which act to exfoliate the skin when rubbed on a user's body. The bar also broadly comprises holding compounds, which hold the exfoliant granules in place prior to use, and then slowly release the granules over a predetermined period, for example approximately 10 minutes when used in the shower. These holding compounds include water soluble granules and a binder.

In one embodiment, the bar may contain water soluble granules and exfoliant granules. The water soluble granules are preferable sucrose. The water soluble granules and exfoliant granules are preferably substantially the same size (e.g. cross sectional diameter of the granules are preferable similar). The applicant has discovered that if the particles are substantially the same size, they can more easily be compressed and held in the compressed shape prior to use.

The binder, which in a preferred embodiment is avena sativa (oat) leaf powder, also known as colloidal oatpowder, binds the granules together and maintains the bar in its compressed form.

The bar also contains emollients such as cocoa seed butter and olive oil. In a preferred embodiment, the bar contains both.

The bar may also contain a parfum. In a preferred embodiment where the exfoliant granules are coffee seed powder, the parfum may be selected to complement the natural smell of the powder. The bar may also contain a colourant.

The bar comprises a substantially uniform mixture of at least the exfoliant granules, water soluble granules and binder, and preferably of all components.

### EXAMPLE 1

In this example, a body scrub according to the invention was prepared using a simple hand operated soap mold.

The components were as follows in the provided proportions:

| Component | Proportion by weight (%) |
|---|---|
| Coffee grounds (exfoliant granules) | 60 |
| Sugar (exfoliant granules) | 10 |
| Salt (exfoliant granules) | 5 |
| Bees Wax (Binder) | 5 |
| Coconut Oil (Emollient) | 20 |
| Silicone (Emollient) | 2 |
| JoJoba Oil (Emollient) | 0.01 |
| Vitamin Oil E (Emollient) | 0.01 |
| Grapeseed Oil (Emollient) | 0.01% |
| Polysorbate (Emulsifier) | 2% |

The dry ingredients were placed into a mixing tank and stirred.

The wet ingredients were placed into a separate mixing tank and heated until melted.

The melted wet ingredients were poured into the dry ingredients and mixed thoroughly

The resultant mixture was placed into moulds and compressed using hand pressure or a power press.

The moulded bars were left to set and dry and then packed.

### EXAMPLE 2

In another example, the bar may consist of the components in the following proportions:

| Component | Proportion by weight (%) |
|---|---|
| Sucrose (such as dark brown sugar) | 47 |
| Coffee seed powder | 27.5 |
| Avena Sativa Leaf Powder (binder) | 15 |
| Cocoa butter | 5 |
| Olive oil | 3 |
| Polymethylsilsesquioxane | 2 |
| Parfum | 0.5 |

The bar of example 2 is made by forming a substantially uniform dry mixture which is then compressed into a substantially cube-type shape. Of course, the bar can be any shape. The bar is formed using a mechanical press or hand press asserting 100 psi and above on the bar.

While the examples provided detail specific % by weight quantities of the components of the composition, it will be evident to the skilled addressee that other % by weight quantities could be used to form a single use exfoliant bar. The choice of % by weight quantities of the components may be impacted by decisions relating the level of exfoliation and comfort required (e.g. high quantities of exfoliant may be included to increase the exfoliation impact of the bar, while relatively high quantities of coffee grounds may be included in lieu of relatively high quantities of sugar and salt to provide an exfoliant bar that is relatively gentle on the skin) and manufacturing cost (e.g. coffee grounds may be more expensive that sugar). The % by weight quantities of the binders will also vary in dependence on the choice of exfoliant.

In use with water, for example in a shower or bath, some of the components of the exfoliant bar will slowly melt or dissolve releasing the exfoliant granules from the bar. In the second example, the sucrose will dissolve to release the exfoliant granules. The slow release of the granules means that a user may apply the bar, including the exfoliant granules, to specific areas for exfoliation. The emollients are usually absorbed by the skin and hydrate the skin, while the polymethylsilsesquixane (also known as Gransil PSQ) makes the user's skin feel smoother.

The word ‘comprising’, and forms of the word ‘comprising’, when used in this specification is taken to specify the presence of stated features, integers, steps or components but does not preclude the presence or addition of one or more other features, integers, steps, components or groups thereof.

In this specification where a document, act or item of knowledge is referred to or discussed, this reference or discussion is not an admission that the document, act or item of knowledge or any combination thereof was at the priority date, publicly available, known to the public, part of the common general knowledge; or known to be relevant to an attempt to solve any problem with which this specification is concerned.

1. A single use soap free body scrub bar comprising a substantially uniform mixture of:
(a) exfoliant granules made of one or more materials selected from the group consisting of: coffee seed powder, sugar, salt, green tea and nut husks; and
(b) one or more binders configured to bind the exfoliant granules together on pressing of the mixture;
wherein, in use with water, the one more binders will slowly release the granules over a predetermined period.

2. (canceled)

3. The single use soap free body scrub bar of claim 1, wherein the exfoliant granules are made of two or more materials and the granules are substantially the same size.

4. The single use soap free body scrub bar of claim 1, further comprising one or more emollients.

5. The single use soap free body scrub bar of claim 1, further comprising a lubricant.

6. The single use soap free body scrub bar of claim 1, further comprising a parfum.

7. The single use soap free body scrub bar of claim 1, further comprising a colourant.

8. (canceled)

9. The single use soap free body scrub bar of claim 1 wherein one of the one or more binders is avena sativa leaf powder.

10. The single use soar free body scrub bar of claim 4 wherein the emollients include one or both of cocoa seed butter and olive oil.

11. (canceled)

12. A soap free exfoliant bar composition comprising exfoliant granules and binding particles, wherein the binding particles are capable of binding the exfoliant granules in the exfoliant bar composition together on pressing of the exfoliant bar composition.

13. The soap free exfoliant bar composition according to claim 12, wherein the composition comprises greater than 50% exfoliant granules by total weight of the composition.

14. The soap free exfoliant bar composition according to claim 12, wherein the exfoliant granules comprises a mixture of components selected from the group consisting of coffee grounds, sucrose particles and salt particles.

15. The soap free exfoliant bar composition according to claim 14, wherein an average particle size of the mixture of exfoliant granules is between 0.5 and 1 mm in diameter, and the average particle size of each of the exfoliant granules is substantially the same.

16. The soap free exfoliant bar composition according to claim 12, wherein the binding particles comprises avena sativa leaf powder particles.

17. The soap free exfoliant bar composition according to claim 16, wherein the composition is in dry powder form.

18. The soap free exfoliant bar composition according to claim 12, wherein the binding particles comprises a wax.

19. The soap free exfoliant bar composition according to 18, wherein the composition is in a wet form.

20. A method of forming a soap free exfoliant bar having a matrix containing exfoliant granules and binding particles, the method comprising the steps of:
providing in a mould a mixture containing the exfoliant bar composition; and
pressing the mixture in a mould to bind the mixture to form the exfoliant bar shaped by the mould.

21. The method according to claim 20, wherein the mixture contains additional binding particles to that contained in the exfoliant bar composition.

22. The method according to claim 20, wherein the mixture contains emollients.

23. (canceled)

24. The method according to claim 20 wherein the mixture is pressed at a pressure of above 100 psi to form the exfoliant bar.

25. (canceled)
